(19) United States
(12) Patent Application Publication
(10) Pub. No.: US 2013/0263853 A1
(43) Pub. Date: Oct. 10, 2013

(54) SPLIT-TYPE CONTROLLING DEVICE FOR PRODUCING OXYGEN AND DELIVERING AIR

(71) Applicant: Fu-Lai HAN, Tainan City (TW)

(72) Inventor: Fu-Lai HAN, Tainan City (TW)

(21) Appl. No.: 13/442,661

(22) Filed: Apr. 9, 2012

(51) Int. Cl. A61M 16/00; B01D 53/02; B01D 50/00; B82Y 30/00

(52) U.S. Cl. 128/204.23; 96/408; 96/118; 977/778

(57) ABSTRACT

The present invention relates to a split-type controlling device for producing oxygen and delivering air and includes an outdoor unit, a plurality of indoor units, and a control panel. The control panel includes a controlling unit, which is utilized to control the outdoor unit and the indoor units. Carrying oxygen produced by the outdoor unit to an indoor space through an oxygen outlet preferably enhances air quality indoors.

### 1. FIELD OF THE INVENTION

The present invention relates to a split-type controlling device for producing oxygen and delivering air, especially to the device that allows an outdoor unit to produce oxygen and delivers the oxygen to indoor units, so that an indoor space could be full of oxygen.

### 2. DESCRIPTION OF THE RELATED ART

An R.O.C. patent No. 566521 discloses “A SPLIT AIR CONDITIONER PROVIDING OXYGEN AND PURIFICATION”. Wherein, an outdoor unit has an oxygen enrichment device to produce oxygen of high concentration. The oxygen is further delivered to a heat exchanger installed in an indoor unit by a pipe, so that the oxygen that contains high temperature and rich moisture is further processed and thence delivered to an indoor space. Accordingly, the indoor space is provided with high oxygen content.

Afore disclosures have shortcomings as follows:

1. The oxygen enrichment device is a subordinate device to the air conditioner. Namely, there is no exclusive and proper pipe arrangement for delivering oxygen, and there is no controlling module. Accordingly, the oxygen is unsuitably delivered.

2. Referring to FIG. 5 of afore disclosure, an oxygen enrichment module of the oxygen enrichment device utilizes an oxygen enrichment membrane to execute a separation of oxygen over nitrogen. Through the molecule membrane, the air is produced with less than 30% oxygen content. Herein, if the air with less than 30% oxygen content is released in the indoor space, it is in fact not helpful to enhance the oxygen content in the indoor space.

3. The air conditioner of the disclosure does not filter the outdoor air first and then deliver clean air to the oxygen enrichment device for generating oxygen. Accordingly, the oxygen provided by the oxygen enrichment device is not clean enough. As a result, the air quality indoors is influenced.

4. The oxygen flow generated in accordance with the oxygen enrichment membrane is less than 10 liters per minute. Obviously, the oxygen flow is unsatisfactory for enhancing the oxygen content indoors.

5. The oxygen generated in the disclosure does not go through a dehydration process. The oxygen is directly delivered to the indoor unit. Wherein, the pipe easily gathers moss in view of humidity. As a result, the oxygen quality is influenced, and the maintenance of the device and the pipe becomes difficult.

### SUMMARY OF THE INVENTION

A split-type controlling device for producing oxygen and delivering air in accordance with the present invention comprises an outdoor unit having an air purification unit, an oxygen condenser, and a molecule sieve that are orderly connected by a pipe; a plurality of indoor units being connected to the molecule sieve through the pipe; each of the indoor units having an oxygen outlet, and an oxygen sensor disposed near the oxygen outlet; and a control panel having a controlling unit; the controlling unit being electrically connected to the outdoor unit and the indoor units so as to control an operation of the outdoor unit and the indoor units; the controlling unit allowing oxygen generated by the outdoor unit to be output from the oxygen outlet of one of the indoor units.

Advantages over the present invention are as follows:

1. The present invention allows a single outdoor unit to be set correspondingly to multiple indoor units. Moreover, the pipe is arranged properly for each indoor unit to be placed in different rooms. Whereby, a supplying mode and a supplying position of the oxygen could be controlled by the control panel, thence conducing to an exclusive device for producing oxygen and delivering the same.

2. When the separation of oxygen over nitrogen in the air is executed by the molecule sieve, the oxygen of high concentration that contains 40% to 50% oxygen could be produced. The flow is also promoted to 40 litters to 50 litters per minute. Accordingly, if such oxygen of high concentration is released in the indoor space, the oxygen content indoors is obviously encouraged.

3. External air is efficiently filtered; therefore, the device produces clean oxygen.

4. The oxygen content can reach to 90% for emergent patients while emergency.

5. The split-type design keeps the indoor space quiet. When Pythoncidere is provided, the air quality indoors is even better.

6. When the control panel is applied for users to choose a manual mode, users are able to manually turn on the outdoor unit as well as the oxygen outlet of any one of the indoor units. Accordingly, the controlling device is more suited to the need of users with economical electricity.

7. When the control panel is further applied for users to choose a semi-automatic mode or an automatic mode, a single controlling means or a united controlling means is accessible. When the single controlling means is adopted, the oxygen sensor automatically enhances or reduces the oxygen concentration according to a predetermined value. When the united controlling means is adopted, a detecting receiver is able to detect and receive a location signal from an emitter. Accordingly, when users approach any detecting receiver, the outdoor unit is automatically turned on so as to produce oxygen. Thereby, the indoor unit set in accordance with where the users stay allows its oxygen outlet to release oxygen. These modes are especially economical of electricity. Moreover, the controlling device for producing oxygen is further intelligent.

### BRIEF DESCRIPTION OF THE DRAWINGS

FIG. 1 is a schematic view showing components and their accordant relationships of the present invention;

FIG. 2 is a schematic view showing a relationship between switches of a control panel and an emitter of the present invention;

FIG. 3 is a schematic view showing a relationship among an outdoor unit, indoor units, and the control panel of the present invention in operation;

FIG. 4 is a schematic view showing a relationship between an emitter and a detecting receiver of the present invention; and

FIG. 5 is a schematic view showing an electroencephalogram detector included in the indoor unit of the present invention.

### DETAILED DESCRIPTION OF THE PREFERRED EMBODIMENTS

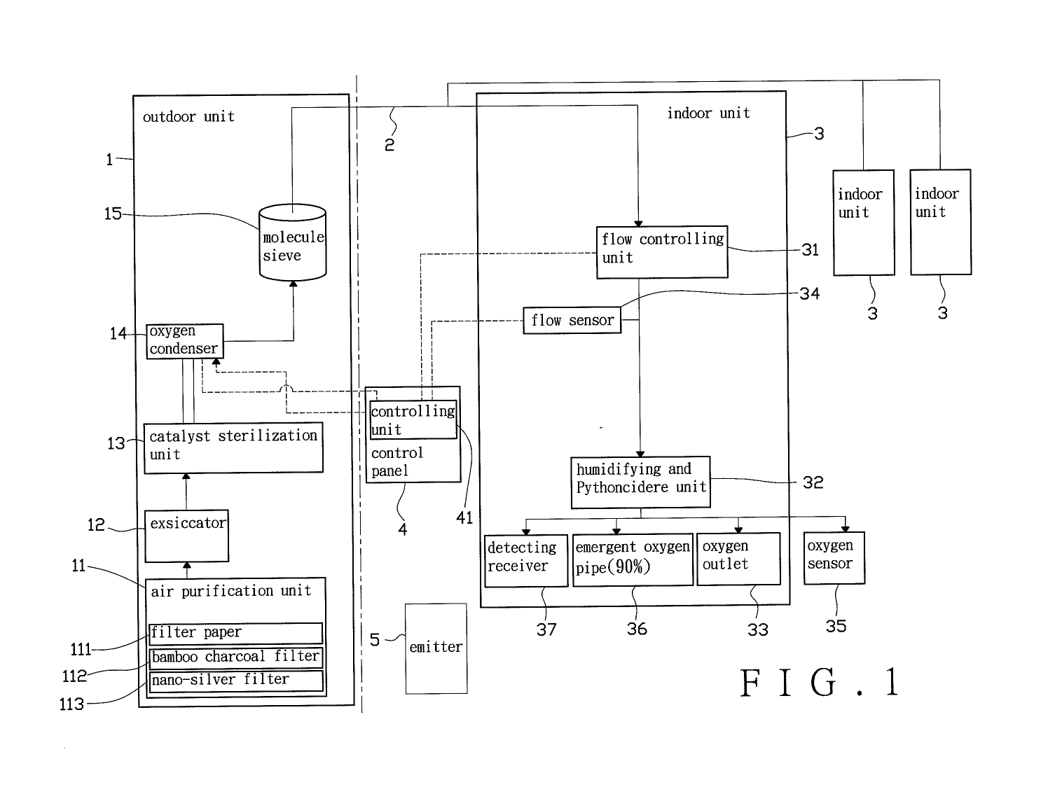

Referring to FIG. 1, a split-type controlling device for producing oxygen and delivering air comprises:

an outdoor unit 1 being installed outdoors; the outdoor unit 1 having an air purification unit 11, an exsiccator 12, a catalyst sterilization unit 13, an oxygen condenser 14, and a molecule sieve 15 that are orderly connected by a pipe 2;

at least one indoor unit 3 being installed indoors; the indoor unit 3 being connected to the molecule sieve 15 through the pipe 2; each of the indoor units 3 having a flow controlling unit 31, a humidifying and Pythoncidere unit 32, an oxygen outlet 33, a flow sensor 34, an oxygen sensor 35 that are connected to the indoor unit 3; and

a control panel 4 having a controlling unit 41; the controlling unit 41 being connected to the outdoor unit 1 and the indoor unit 3 so as to control an operation of the outdoor unit 1 and the indoor unit 3.

Wherein, the outdoor unit 1 allows external air to enter the air purification unit 11. Herein, the air purification unit 11 provides three filtering means. A piece of filter paper 111 blocks heavy metal or large dust particles in the air first. Then, a bamboo charcoal filter 112 deodorizes, dehydrates, and filters the air. Next, a nano-silver filter 113 utilizes the nano-silver to achieve antiseptic, deodorized, and anticorrosive functions. Accordingly, by means of the three filters, cleaner air is easily obtainable.

The air that is purified by the air purification unit 11 enters the exsiccator 12 via the oxygen condenser 14, so that moisture contained in the air is eliminated. Subsequently, the dehydrated air enters the catalyst sterilization unit 13 for being processed with photo catalyst or low-temperature catalyst. Herein, both the photo catalyst and the low-temperature catalyst have a high-efficiency broad spectrum. Therefore, such catalysts are good for sterilization and disinfection.

Further, the sterilized air enters the molecule sieve 15. Herein, the nitrogen whose particle size is larger is blocked from the molecule sieve 15. As to the oxygen, the molecule sieve does not block it since the oxygen has a smaller particle size. Therefore, the oxygen continues going forward for resulting in oxygen of high concentration. In this embodiment, there are twelve molecule sieve tubes defined on the molecule sieve 15 for respectively and orderly producing the oxygen of high concentration. As a result, the oxygen with a high flow rate and high concentration is available.

The indoor unit 3 carries the oxygen via the pipe 2, controls the flow rate via the flow controlling unit 31, and monitors the flow rate via the flow sensor 34. The humidifying and Pythoncidere unit 32 is utilized for adjusting relative humidity in the indoor space. Namely, the humidifying and Pythoncidere unit 32 could be switched to a slight mode, a medium mode, or a heavy mode. Moreover, under a certain temperature, the relative humidity should be maintained between 40% and 60%. To do so, a fine spray for increasing humidity enters the pipe 2, the fine spray further mixes with the oxygen, and the oxygen containing moisture is released by means of the oxygen outlet 33 to the indoor space.

Additionally, there is an air hole along with an oil can defined on the humidifying and Pythoncidere unit 32. Whereby, when the oxygen is carried to the air hole, the smell of Pythoncidere is also released to the indoor space. While an emergent oxygen pipe 36 is provided on one indoor unit 3, the oxygen content can reach to 90% for emergent patients in time of emergency.

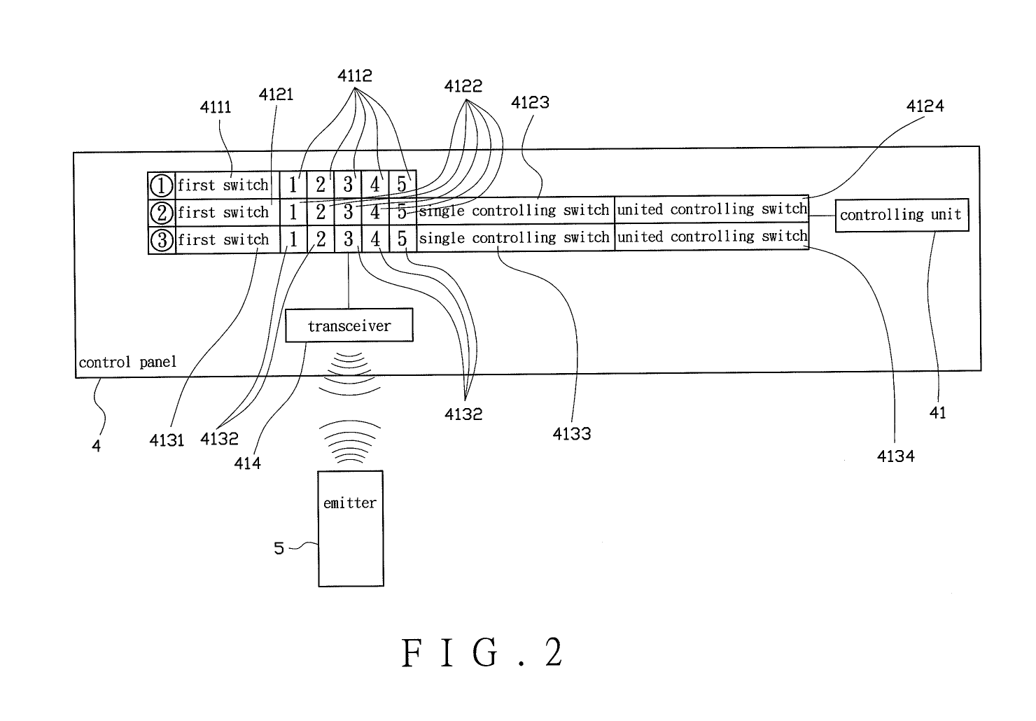

Referring to FIG. 2, the control panel 4 includes a controlling unit 41 that provides a manual mode 411, a semi-automatic mode 412, and an automatic mode 413. The manual mode 411 has a first switch 4111 for the outdoor unit and five second switches 4112 for the oxygen outlets. The semi-automatic mode 412 has a first switch 4121 for the outdoor unit, five second switches 4122 for the oxygen outlets, a single controlling switch 4123, and a united controlling switch 4124. The automatic mode 413 has a first switch 4131 for the outdoor unit, five second switches 4132 for the oxygen outlets, a single controlling switch 4133, and a united controlling switch 4134. The control panel 4 further includes a transceiver 414, which is electrically connected to the switches correlated to the manual mode 411, the semi-automatic mode 412, and the automatic mode 413, and an emitter 5 disposed correspondingly to the transceiver 414. In this embodiment, the emitter 5 adopts a remote control that generates a selective signal to the transceiver 414, thereby allowing the manual mode 411, the semi-automatic mode 412, or the automatic mode 413 to be adopted. Afore design allows users to freely choose their desiring mode by manual or by remote operation.

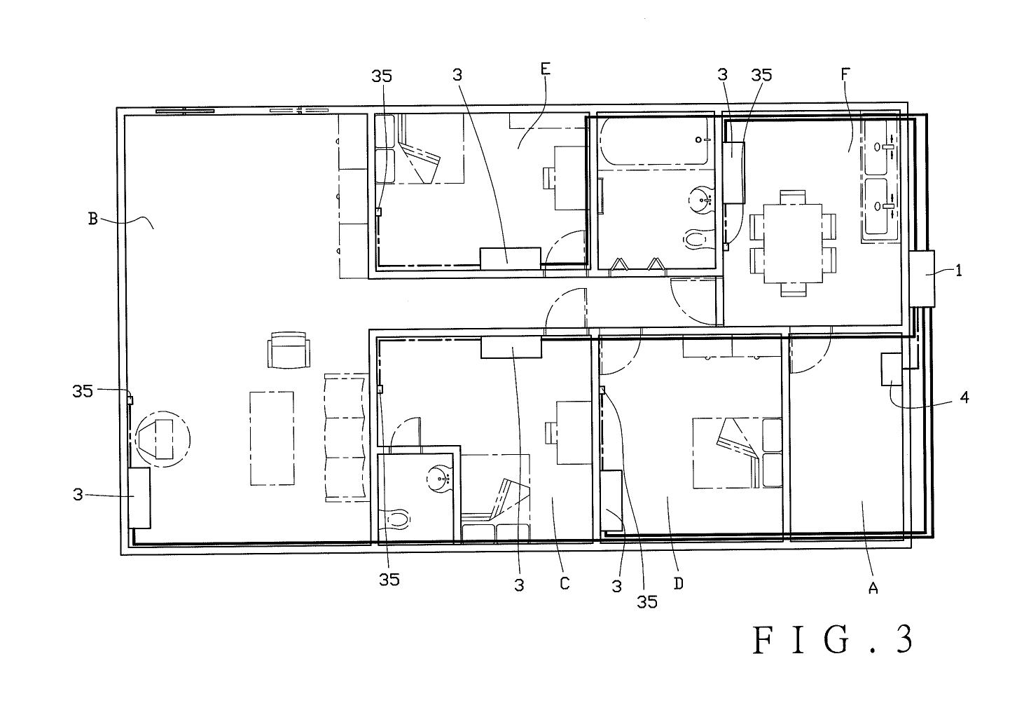

Referring to FIGS. 1 and 3, the control panel 4 is installed in a storeroom A. The five indoor units 3 with their respective oxygen outlets 33 are placed in a living room B, a master bedroom C, a first bedroom D, a second bedroom E, and a dinning room/kitchen F. When the control panel 4 is just turned on by either the manual operation or the remote operation, and when no mode is adopted, the oxygen produced by the outdoor unit 1 will be output by the oxygen outlet 33 from the indoor unit 3 in the living room B. When users choose the single controlling switches 4123, 4133 of the semi-automatic mode 412 or the automatic mode 413, the oxygen sensor 35 connected to the indoor unit 3 is accordingly triggered. Basically, the oxygen content of the air is about 20.6% to 20.9%. Indoors, the preferred oxygen content of the air is 21% to 30%. Herein, the controlling unit 41 is able to set appropriate maximum and minimum of the oxygen content. Additionally, when the oxygen sensor 35 senses, a feedback signal is transmitted to the controlling unit 41, so that the accordant oxygen outlet 33 gives oxygen to the indoor space. The control panel 4 provides two options for setting the oxygen content. The first option is to set the oxygen content for health care. Herein, the oxygen content of the first option is similar to the oxygen content of a green shower in nature. Namely, the oxygen sensor 35 helps maintain the oxygen concentration between 21% and 24%, which is suited to normal people. The second option is to set the oxygen content for medical care. Herein, the oxygen concentration in this option is maintained between 24% and 30%, which is suited to patients having pathological hypoxia. Once either option is decided, the oxygen sensor 35 emits a signal to the controlling unit 41 for switching on the flow controlling unit 31 to give oxygen when the oxygen concentration is below the minimum. If the oxygen concentration is over the maximum, the oxygen is not supplied. Accordingly, the automatic mode is achieved.

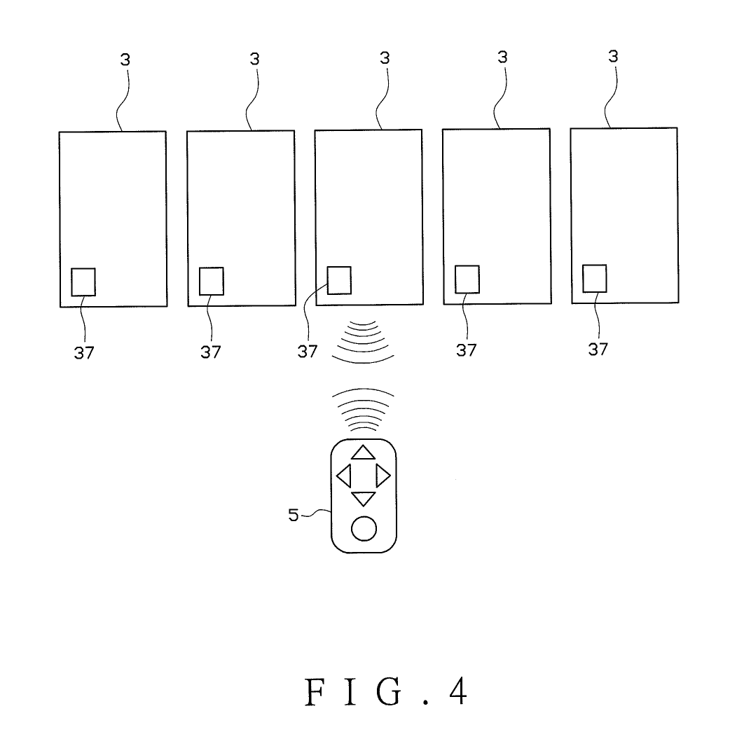

Referring to FIG. 4, when users turn on the united controlling switches 4123, 4134 of the semi-automatic mode 412 and the automatic mode 413, both the oxygen sensor 35 and a detecting receiver 37 are concurrently triggered. Accordingly, the detecting receiver 37 detects and receives a location signal from the emitter 5 for automatically turning on or turning off the oxygen outlets 33 in the rooms, which is more economical of electricity.

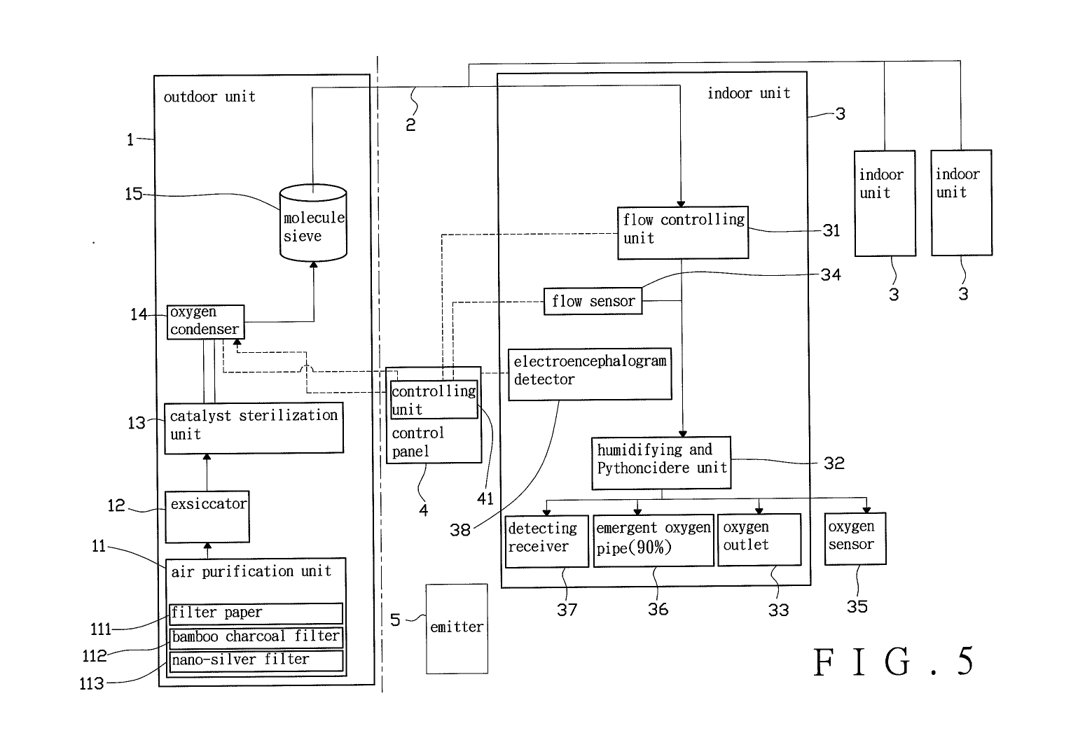

Referring to FIG. 5, each of the indoor units 3 includes an electroencephalogram detector 38 for detecting electroencephalogram of human body indoors and transforming an α value and a β value of the electroencephalogram to electronic signals. Since human body generates different electroencephalograms while receiving diverse oxygen concentration, it could be easily realized that if the present oxygen concentration is suited to the human body. Whereby, the control panel 4 adjusts the output oxygen concentration in accordance with the electronic signals. Therefore, the oxygen concentration indoors is indeed suited to human body.

I claim:

1. A split-type controlling device for producing oxygen and delivering air comprising:
an outdoor unit having an air purification unit, an oxygen condenser, and a molecule sieve that are orderly connected by a pipe;
a plurality of indoor units being connected to said molecule sieve through said pipe; each of said indoor units having an oxygen outlet, and an oxygen sensor disposed near said oxygen outlet; and
a control panel having a controlling unit; said controlling unit being electrically connected to said outdoor unit and said indoor units so as to control an operation of said outdoor unit and said indoor units; said controlling unit allowing oxygen generated by said outdoor unit to be output from said oxygen outlet of one of said indoor units.

2. The split-type controlling device as claimed in claim 1, wherein, said air purification unit includes a piece of filter paper, a bamboo charcoal filter, and a nano-silver filter

3. The split-type controlling device as claimed in claim 1, wherein, said indoor units include a flow sensor.

4. The split-type controlling device as claimed in claim 1, wherein, said indoor units include an emergent oxygen pipe.

5. The split-type controlling device as claimed in claim 1, wherein, said outdoor unit includes an exsiccator connected between said air purification unit and said oxygen condenser through said pipe.

6. The split-type controlling device as claimed in claim 5, wherein, said outdoor unit includes a catalyst sterilization unit connected between said exsiccator and said oxygen condenser.

7. The split-type controlling device as claimed in claim 1, wherein, said indoor units include a flow controlling unit.

8. The split-type controlling device as claimed in claim 1, wherein, said indoor units include a humidifying and Pythoncidere unit.

9. The split-type controlling device as claimed in claim 1, wherein, said control panel includes a manual mode, a semi-automatic mode, and an automatic mode.

10. The split-type controlling device as claimed in claim 9, wherein, said manual mode has a first switch for said outdoor unit and a plurality of second switches for said oxygen outlets.

11. The split-type controlling device as claimed in claim 9, wherein, said semi-automatic mode has a first switch for said outdoor unit, a plurality of second switches for said oxygen outlets, a single controlling switch, and a united controlling switch.

12. The split-type controlling device as claimed in claim 9, wherein, said automatic mode has a first switch for said outdoor unit, a plurality of second switches for said oxygen outlets, a single controlling switch, and a united controlling switch.

13. The split-type controlling device as claimed in claim 9, wherein, said control panel includes a transceiver and an emitter generating a selective signal to said transceiver, thereby allowing said manual mode, said semi-automatic mode, or said automatic mode to be selectively operated.

14. The split-type controlling device as claimed in claim 13, wherein, each indoor unit includes a detecting receiver for detecting and receiving a location signal from said emitter.

15. The split-type controlling device as claimed in claim 13, wherein, each of said indoor units includes an electroencephalogram detector for detecting electroencephalogram of human body and transforming said detection to electronic signals, thereby allowing said control panel to adjust oxygen concentration according to said electronic signals.
